(19) United States
(12) Patent Application Publication
(10) Pub. No.: US 2015/0247874 A1
(43) Pub. Date: Sep. 3, 2015

(54) METAL NANOPARTICLE-APTAMER CONJUGATES FOR DETECTION OF SMALL MOLECULES AND IN-THE-FIELD USE THEREOF

(71) Applicant: Government of the United States as Represented by the Secretary of the Air Force, Wright-Patterson AFB, OH (US)

(72) Inventors: Jorge Luis Chavez Benavides, Kettering, OH (US); Joshua Smith, Dayton, OH (US); Nancy Kelley-Loughnane, Fairfield, OH (US); Joshua Hagen, Cincinnati, OH (US); Peter A. Mirau, Dayton, OH (US); Rajesh Naik, Centerville, OH (US)

(73) Assignee: Government of the United States as Represented by the Secretary of the Air Force, Wright-Patterson AFB, OH (US)

(21) Appl. No.: 14/193,673

(22) Filed: Feb. 28, 2014

(51) Int. Cl. G01N 33/94

(52) U.S. Cl. CPC G01N 33/946; B82Y 20/00

(57) ABSTRACT

A method of detecting a presence of an analyte in a sample. The method includes selecting an aptamer selective to the analyte and binding the aptamer to a nanoparticle. The nanoparticles having a free state are perceived as a first color and an aggregate state are perceived as a second color. The sample is introduced to the nanoparticle-bound aptamers, and aggregation of the nanoparticles is promoted. A colorimetric change is analyzed, wherein the aggregate state of the nanoparticles is achieved in the presence of the analyte in the sample.

### RIGHTS OF THE GOVERNMENT

The invention described herein may be manufactured and used by or for the Government of the United States for all governmental purposes without the payment of any royalty.

### FIELD OF THE INVENTION

The present invention relates generally to biorecognition and, more particularly, to colorimetric biorecognition sensor systems.

### BACKGROUND OF THE INVENTION

Biorecognition is that portion of a biosensor system configured to exhibit affinity phenomenon to a target analyte. When the target analyte is present, the biosensor system converts the biochemical signal to signals of another type, for example, electrical or optical. Nanomaterials have revolutionized the area of biosensor development by introducing materials having many shapes and sizes. This adjustability offers a plurality of sensing platforms for different applications.

Thus, the combination of nanomaterials with biorecognition elements has enabled hybrid sensing materials having great target specificity, selectivity, and tunable outputs, including, for example, colorimetric outputs, electronic outputs, and so forth. For example, metal nanoparticles exhibit size-dependent color changes, which may be exploited for the design of colorimetric detection systems. More specifically, gold nanoparticles (“AuNPs”) are perceived with a red color when dispersed in solution and a blue color when aggregated. The transition from the dispersed state (perceived red) to the aggregated state (perceived blue) has been engineered to be in response to an external stimulus.

Despite all these advantages and the great promise of nanomaterials in developing bio-sensing systems, the feasibility of using such systems remains to be demonstrated. In the case of colorimetric sensors, the integration of automated analysis tools that simplify data analysis for on-spot decision making is critical to help transitioning this technologies to the field. Quantification of color change analysis is a simple task in a laboratory setting, where a spectrometer is available; however, it would be advantageous to have a fast and reliable test, for use in the field, that can determine whether the chemical entity of an unknown specimen (for example, does a white powder comprise cocaine). It would not be necessary for such assays to be highly sensitive as the substances to be tested are often available in significant quantities.

Moreover, relatively little is known of the interactions between aptamers and the AuNPs used in colorimetric analysis. Conventionally it has been thought that the aptamer, have a particular conformation, binds to the AuNP in a fast exchange. The binding event is believed to then lead to a partial unfolding of the aptamer, which leads to rapid exchange of selected imino protons. According to this conventionally understood mechanism, the number of available aptamers available to colorimetric analysis is limited to those having particular conformational changes.

As a result, there remains a need for a nanoparticle-based colorimetric sensor, utilizing a wider range of aptamers, for in-the-field use.

### SUMMARY OF THE INVENTION

The present invention overcomes the foregoing problems and other shortcomings, drawbacks, and challenges of implementing nanoparticle-based colorimetric sensors for in-the-field use. While the invention will be described in connection with certain embodiments, it will be understood that the invention is not limited to these embodiments. To the contrary, this invention includes all alternatives, modifications, and equivalents as may be included within the spirit and scope of the present invention.

According to one embodiment of the present invention a method of detecting a presence of an analyte in a sample includes selecting an aptamer selective to the analyte and binding the aptamer to a nanoparticle. The nanoparticles having a free state are perceived as a first color and an aggregate state are perceived as a second color. The sample is introduced to the nanoparticle-bound aptamers, and aggregation of the nanoparticles is promoted. A colorimetric change is analyzed, wherein the aggregate state of the nanoparticles is achieved in the presence of the analyte in the sample.

In some aspects of the invention, analyzing the colorimetric change includes loading an image of the sample and determining a red-value, a blue-value, and a green-value for a portion of the image representing the sample. The red-, blue, and green-values are converted to CIE color space coordinates and determined whether the CIE color space coordinates are within a range defined by a low concentration of the analyte and a high concentration of the analyte. Based on the determination, a positive result for the presence of the analyte is returned when the CIE color space coordinates are within the range or a negative result for the presence of the analyte is returned when the CIE color space coordinates are not within the range.

Still yet another embodiment of the present invention is directed to preparing the sample using an aptamer-nanoparticle conjugate assay and loading an image of the prepared sample. A red-value, a blue-value, and a green-value for a portion of the image representing the prepared sample and are converted to CIE color space coordinates. There is a determination as to whether the CIE color space coordinates are within a range defined by a low concentration of the analyte and a high concentration of the analyte. Based on the determination, a positive result for the presence of the analyte is returned when the CIE color space coordinates are within the range or a negative result for the presence of the analyte is returned when the CIE color space coordinates are not within the range.

According to yet another embodiment of the present invention, an apparatus for detecting a presence of an analyte in a sample includes a camera, a memory, a processor, a display, and program code resident in the memory. The program code is configured to be executed by the processor to acquire a photograph of the sample prepared using an aptamer-nanoparticle conjugate assay and return a result indicating the presence of the analyte. In that regard, the program code determines a red-value, a blue-value, and a green-value for a portion of the loaded image representing the prepared sample and converts the red-value, the blue-value, and the green-value to CIE color space coordinates. The program code then determines whether the CIE color space coordinates are within a range defined by a lowest possible concentration of the analyte and a highest possible concentration of the analyte. Based on the determination, a positive result for the presence of the analyte is returned when the CIE color space coordinates are within the range or return a negative result for the presence of the analyte is returned when the CIE color space coordinates are not within the range.

According to another embodiment of the present invention a method of detecting a presence of an analyte in a sample includes selecting an aptamer selective to the analyte and mixing the aptamer with nanoparticles such at least one aptamer binds to each nanoparticle. The nanoparticles having a free state are perceived as a first color and an aggregate state are perceived as a second color. The sample is introduced into the aptamer and nanoparticle mixture, and aggregation of the nanoparticles is promoted. A colorimetric change is analyzed, wherein aggregate state of the nanoparticles is achieved in the presence of the analyte in the sample.

Additional objects, advantages, and novel features of the invention will be set forth in part in the description which follows, and in part will become apparent to those skilled in the art upon examination of the following or may be leaned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flowchart illustrating an assay in accordance with one embodiment of the present invention.

FIGS. 2A and 2B are representations of MN6 (SEQ ID NO. 2), a cocaine binding aptamer, in unbound and bound states, respectively.

FIG. 3 schematically illustrates the method of FIG. 1.

FIG. 3A schematically illustrates a mechanism for the colorimetric response associated with the mixture of an aptamer, the analyte, and gold nanoparticles.

FIGS. 4A and 4B are flowcharts illustrating a method of colorimetric analysis for the method of FIG. 1 and in accordance with an embodiment of the present invention.

FIG. 5 is a schematic representation of a computer configured to perform the method of FIG. 4B.

FIG. 6 is a screen capture of an exemplary app configured to perform the method of FIGS. 4A and 4B.

FIG. 7 is a flowchart illustrating an assay in accordance with another embodiment of the present invention.

FIGS. 8A and 8B are representations of MN4 (SEQ ID NO. 1), a cocaine binding aptamer, in unbound and bound states, respectively.

FIG. 9 schematically illustrates the method of FIG. 7.

FIG. 9A schematically illustrates a mechanism for the colorimetric response associated with the mixture of an aptamer, the analyte, and gold nanoparticles.

FIGS. 10 and 11 are graphical representations of the aggregation of nanoparticles in response to assays carried out in accordance with embodiments of the present invention.

FIGS. 12A-12D are TEM images of nanoparticles in response to assays, challenged by cocaine, methanol, ecgonine methyl ester hydrochloride, and JWH-018, respectively, and carried out in accordance with embodiments of the present invention.

FIG. 13 is a graphical representation shelf-life studies carried out on assays according to embodiments of the present invention.

FIGS. 14 and 15 are graphical representations of aggregation of nanoparticles in response to assays carried out with conventional drug cutting agents and fillers.

FIG. 16 is a graphical representation of exemplary cocaine calibration curves monitored by conventional plate reader and an embodiment of the method of FIG. 4B.

FIG. 17 is a graphical representation of exemplary cocaine calibration curves monitored by a CIE color analysis method and according to an embodiment of the present invention.

FIG. 18 is a graphical representation of the distribution of exemplary CIE x- and y-coordinates, generated in accordance with an embodiment of the method of FIG. 4B.

FIG. 19 is a graphical representation of the distribution of exemplary CIE x-coordinates, generated in accordance with an embodiment of the method of FIG. 4B.

FIG. 20 is a graphical representation of colorimetric assay results generated according to a conventional plate reader analysis.

FIGS. 21-23 are exemplary NMR spectra of mixtures of gold nanoparticles, cocaine binding aptamers, and cocaine demonstrating the interactions thereof.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

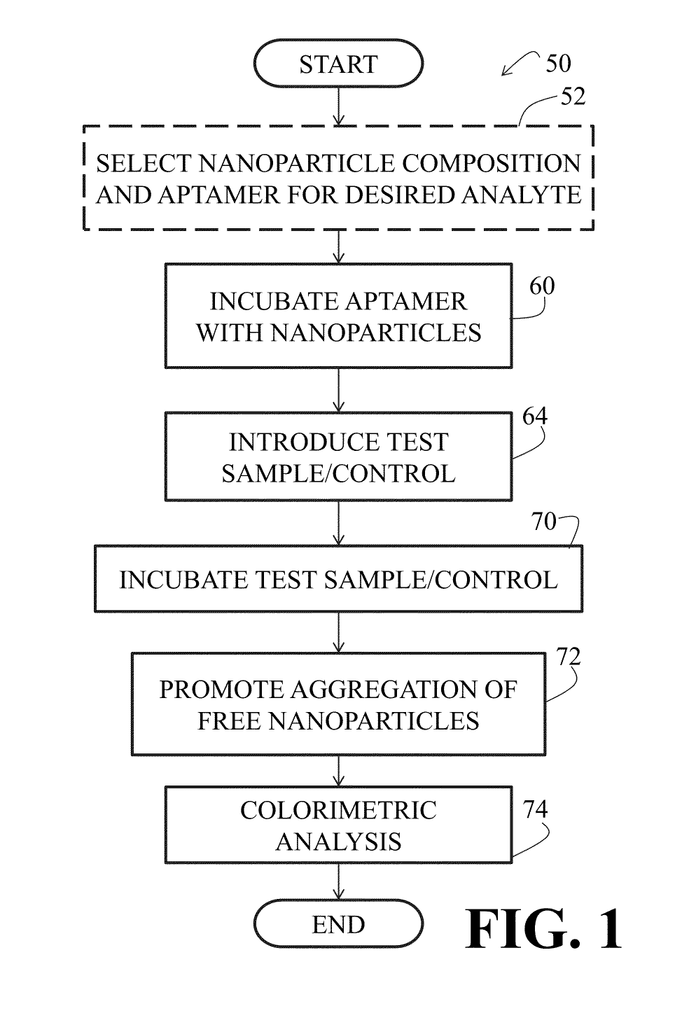

Turning now to the figures, and in particular to FIG. 1, a flowchart 50 illustrating a method of using gold nanoparticle-aptamer conjugates for detection of a desired analyte (or other small molecules) according to one embodiment of the present invention is shown. At start, and if necessary, a composition for nanoparticles may be selected (Block 52). The nanoparticles may comprise a metal and having a diameter that is less than about 100 nm, but are generally configured to undergo a colorimetric change with aggregation. Exemplary nanoparticles may include, for example, gold nanoparticles, wherein synthesis of the gold nanoparticles includes heating and refluxing HAuCl₄, and thereafter adding a sodium citrate solution. The resulting solution may be cooled and filtered to isolate gold nanoparticles.

Also at block 52, an aptamer for the desired analyte may be selected. In that regard, the selected aptamer should be configured to selectively bind the analyte by undergoing a structural change (i.e., an analyte-binding conformation) and without binding analyte derivatives or metabolites. Folded DNA and RNA aptamers with a high affinity and selectivity for target molecules can be selected from in vitro random sequence libraries and are of great interest as the active element in sensors. Aptamers generally comprise DNA or RNA sequences with hairpin folds, stabilized by base paired into stems, which may further fold into compact structures. The folded aptamer structure, bound to a respective target, is the most thermodynamically stable state conformation; the unbound aptamer may be either partially or completely unfolded.

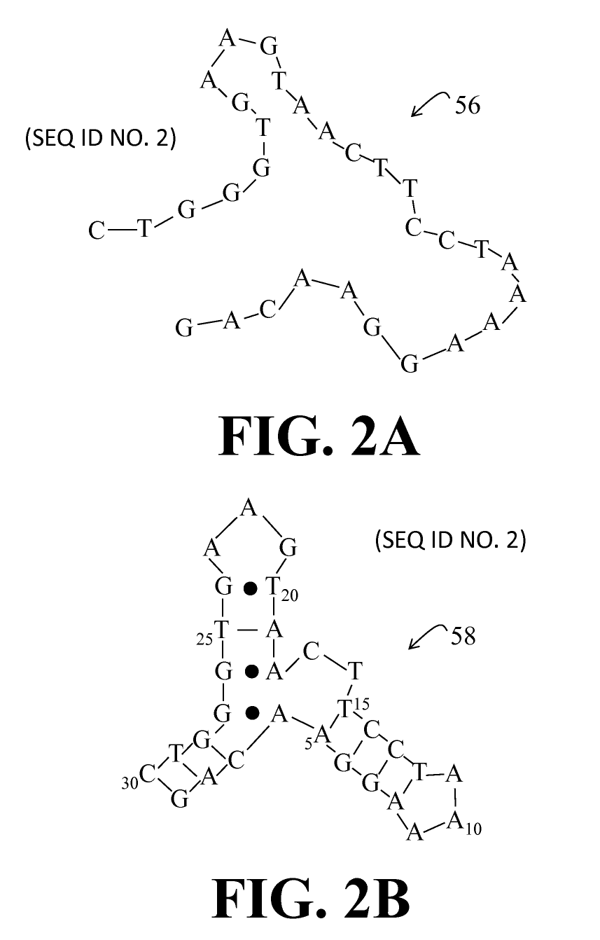

For purposes of illustration only, the method of FIG. 1 is described herein as applied to the use of a cocaine-binding aptamer (“CBA”), specifically, MN6 (SEQ ID NO. 2) (molecular weight being 9288.1 Da). The MN6 (SEQ ID NO. 2) aptamer clone may undergo a transition from an open, single strand conformation 56 (FIG. 2A) to a closed, double-strand conformation 58 (FIG. 2B) when binding to the analyte (here, cocaine). According to still other embodiments of the present invention, the selected aptamer may be configured to undergo still another structural conformational change when binding to the nanoparticle. It will be appreciated by those of ordinary skill in the art that other aptamers may be used so long as the selected aptamer selectively binds the desired analyte. For example, and as described in detail below, the aptamer MN4 (SEQ ID NO. 1) may be used with respect to cocaine analysis. Moreover, it should be noted that a significant conformational change between bound and unbound states of the aptamer.

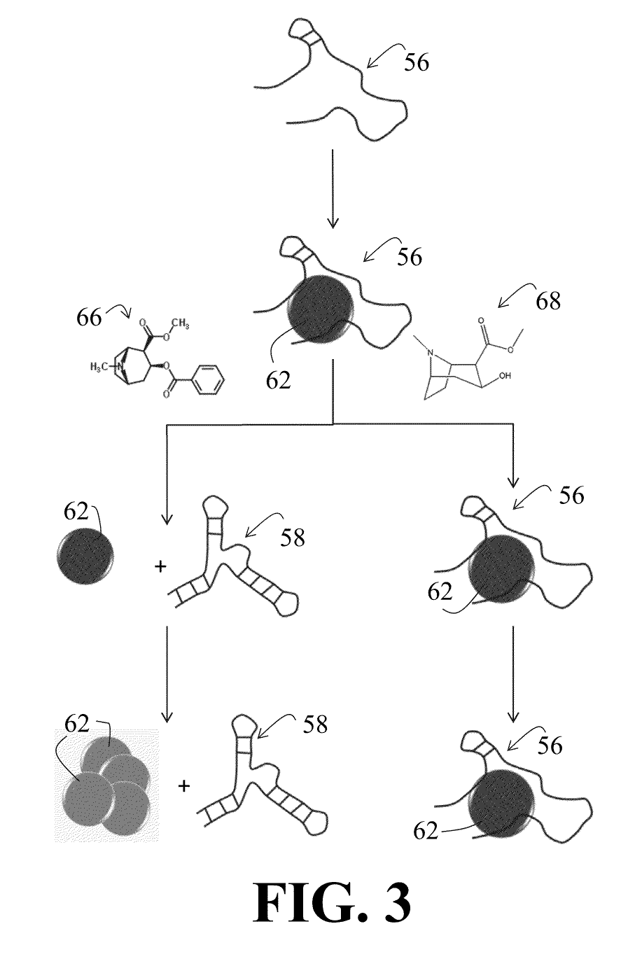

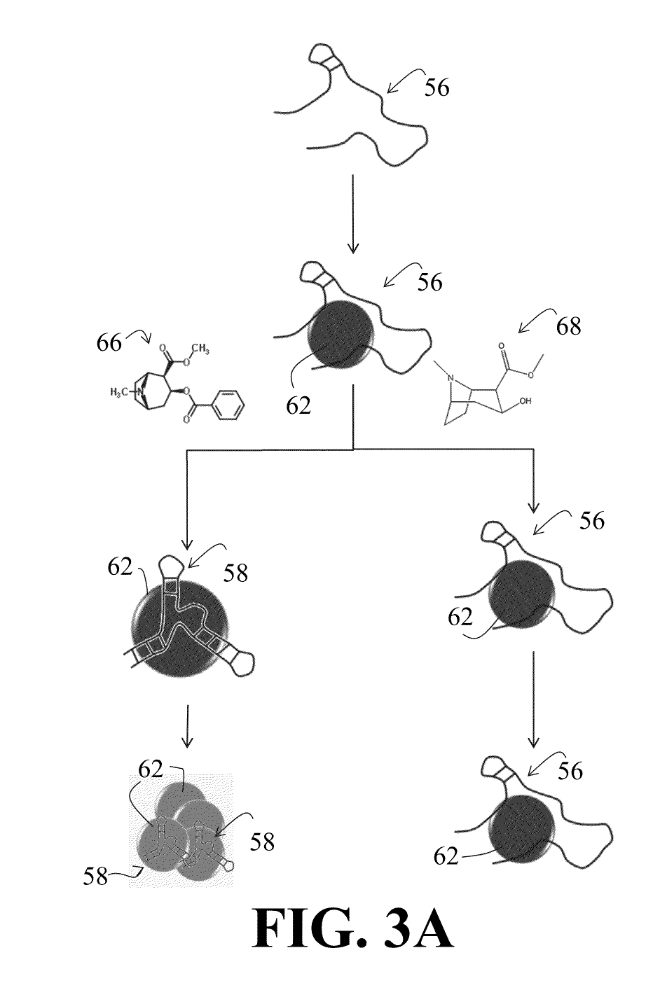

In accordance with block 60 of FIG. 1, and with reference now to FIG. 3, the selected aptamer 56 is incubated with the selected nanoparticles 62 (hereafter, “solution comprising nanoparticle bound aptamer”). If the aptamer 56 is of the type having a structural conformation that binds the nanoparticles 62, binding of one or more aptamers 56 to each nanoparticle 62 may occur. In some embodiments, a ratio of bound aptamer to nanoparticle may be greater than 60 to 1. In any event, incubation (Block 60) may proceed for an incubation period, such as 30 min., or as appropriate or necessary to ensure sufficient binding of at least one aptamer 56 to each nanoparticle 62.

In block 64 of FIG. 1, and as shown in FIG. 3, a test sample is introduced into the incubated solution comprising nanoparticle bound aptamer. The test sample may comprise a biological sample acquired from a patient, an unknown chemical entity, a positive control comprising the analyte of known concentration, or a negative control comprising a known chemical makeup and concentration and without the analyte. For purposes of illustration the unknown chemical entity and the positive control are represented as the cocaine chemical structure 66 in FIG. 3, and the negative control is represented as the ecgonine methyl ester hydrochloride (“EME”) chemical structure 68 in FIG. 3, wherein EME is a cocaine metabolite. The biological sample and the unknown chemical entity may at least partially comprise the desired analyte 66 such that, if present, would be detected. The biological sample may include, for example, urine, saliva, blood, hair, or sweat. If necessary, the biological sample may be prepared in accordance with a sample preparation protocol or used as collected.

Regardless of the source, or identity, each test sample 66, 68 is prepared for assay, which may include, for example, one or more of dissolution, suspension, dilution with a solvent or buffer, or centrifugation.

With the test sample 66, 68 prepared and introduced into the solution of nanoparticle bound aptamer (hereafter, the “test solution”), the test solution may, optionally, incubate for a time, for example, more than 4 hrs (Block 70). If the analyte 66 is present, during the incubation time, the aptamer 56 may undergo a conformational change so as to be released from the nanoparticle 62 and selectively bind the analyte 66, and as is shown in the test sample branch of FIG. 3. Otherwise, and as shown in the negative control branch of FIG. 3, when no analyte 66 is present, the aptamer 56 does not undergo the conformational change and remains bound to the nanoparticle 62.

Alternatively, and as shown in FIG. 3A, aggregation of the nanoparticles 62 may occur within the test sample 66, 68 without liberation of the aptamer 56 from the nanoparticle 62.

In block 72 of FIG. 1, aggregation of the free nanoparticles (that is, nanoparticle having no bound aptamer) is promoted. According to one embodiment of the present invention, aggregation may occur by masking charges of the nucleic acids comprising the aptamer by introducing a salt to the test solution such that the ionic strength of the test solution is increased. As shown in FIG. 3, free nanoparticles aggregate (as shown in the test sample branch of FIG. 3), which may be perceived as a color change. The color change may be, for example, a visible color; however, any detectable change in wavelength (infrared, ultraviolet, etc.) may also be appropriate. Nanoparticles 62 in solutions comprising nanoparticle bound aptamer (as shown in the negative control branch of FIG. 3), resist aggregation and no color change is perceived.

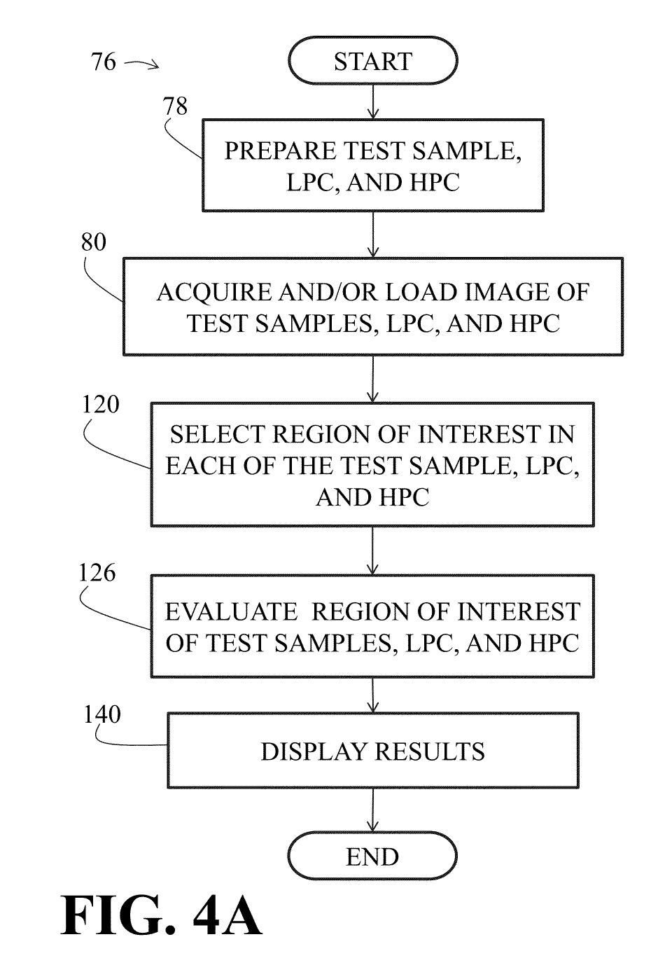

Presence of the color change, or quantification of the amount of color change, may then be determined by a colorimetric analysis (Block 74 of FIG. 1). In that regard, and with reference now to FIG. 4, a flowchart 76 illustrating a method of colorimetric analysis (Block 74 of FIG. 1), and thus determining presence of the desired analyte, is shown in accordance with one embodiment of the present invention. In that regard, a test sample and at least two positive controls are prepared (Block 78). A first of the at least two positive controls comprises a lowest positive concentration (“LPC”) of the desired analyte, and a second of the at least two positive controls comprises a highest positive concentration (“HPC”) of the desired analyte. While sample preparation of Block 78 may be carried out as desired, use of a conventional multi-well plate may be advantageous, as set forth in detail below. Preparation of LPC and HPC along with the test sample is beneficial in that colorimetric analysis (Block 74 of FIG. 1) may be performed under similar conditions, such as room lighting.

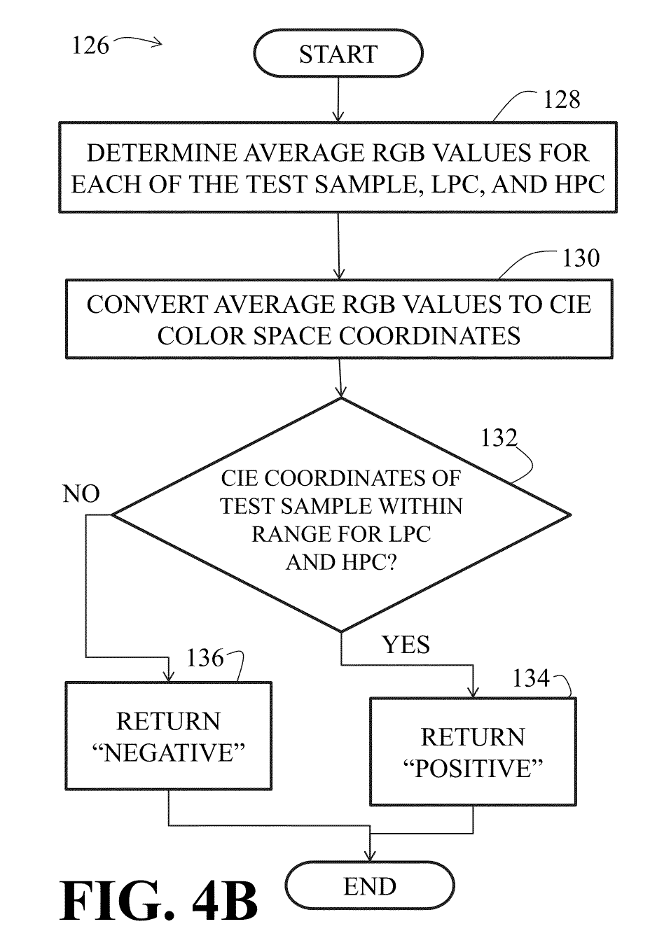

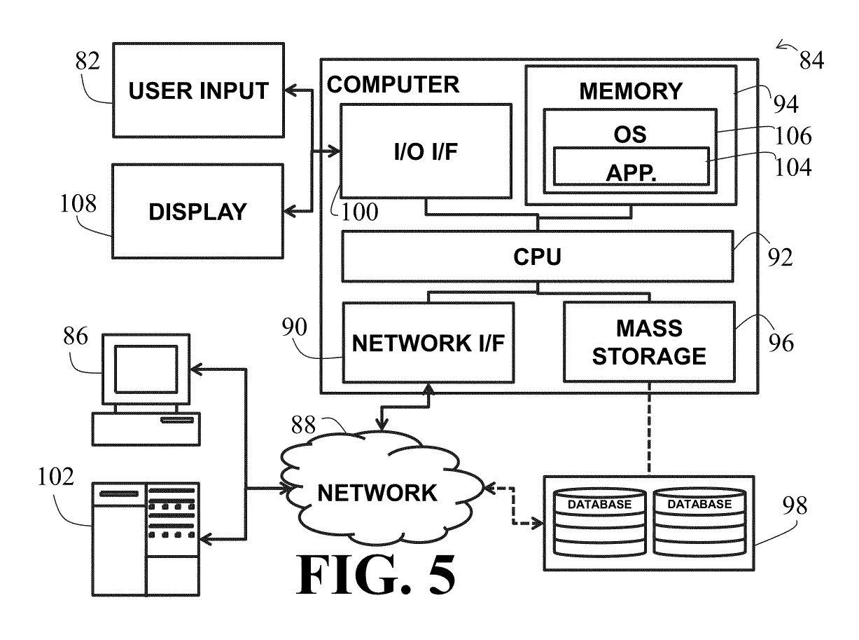

With the test sample, LPC, and HPC prepared (collectively referred to as “the prepared samples”), an image 79 (FIG. 6) of the prepared samples may be acquired, loaded for analysis, or both (Block 80). The image 79 (FIG. 6) may be acquired with an image acquisition device 82 (FIG. 5) in the form of a digital photograph using a digital camera (including digital cameras incorporated as hardware of a smart phone), as a film photograph for developing, or scanned using, for example, a flatbed scanner. In some embodiments, backlighting may be used to minimize variations due to ambient lighting.

In any event, the image 79 (FIG. 6) of the prepared sample is loaded into a computing device 84 (FIG. 5) for the colorimetric analysis (Block 74 of FIG. 1). According to some embodiments of the present invention, the computing device 84 (FIG. 5) may incorporate, or be associated with, the image acquisition device 82.

An exemplary computing device 84 is illustrated in FIG. 5, which is considered to represent any type of computer, computer system, computing system, server, disk array, or programmable device such as multi-user computers, single-user computers, handheld devices (such as a smart phone), networked devices, or embedded devices, etc. The computing device 82 may be implemented with one or more networked computers 86 using one or more networks 88, e.g., in a cluster or other distributed computing device 84 through a network interface (illustrated as “NETWORK I/F” 90). The computing device 84 will be referred to as “computer” 84 for brevity's sake, although it should be appreciated that the term may also include other suitable programmable electronic devices consistent with embodiments of the invention.

The computer 84 typically includes at least one processing unit (illustrated as “CPU” 92) coupled to a memory 94 along with several different types of peripheral devices, e.g., a mass storage device 96 with one or more databases 98, an input/output interface (illustrated as “I/O I/F” 100), and the Network I/F 90. The memory 94 may include dynamic random access memory (“DRAM”), static random access memory (“SRAM”), non-volatile random access memory (“NVRAM”), persistent memory, flash memory, at least one hard disk drive, and/or another digital storage medium. The mass storage device 96 is typically at least one hard disk drive and may be located externally to the computer 84, such as in a separate enclosure or in one or more networked computers 86, one or more networked storage devices (including, for example, a tape or optical drive), and/or one or more other networked devices (including, for example, a server 102).

The CPU 92 may be, in various embodiments, a single-thread, multi-threaded, multi-core, and/or multi-element processing unit (not shown) as is well known in the art. In alternative embodiments, the computer 84 may include a plurality of processing units that may include single-thread processing units, multi-threaded processing units, multi-core processing units, multi-element processing units, and/or combinations thereof as is well known in the art. Similarly, the memory 94 may include one or more levels of data, instruction, and/or combination caches, with caches serving the individual processing unit or multiple processing units (not shown) as is well known in the art.

The memory 94 of the computer 84 may include one or more applications (illustrated as “APP.” 104), or other software program, which are configured to execute in combination with the Operating System (illustrated as “OS” 106) and automatically perform tasks necessary for operating the image acquisition device 82 and/or performing the colorimetric analysis (Block 74 of FIG. 1), with or without accessing further information or data from the database(s) 98 of the mass storage device 96.

Those skilled in the art will recognize that the environment illustrated in FIG. 5 is not intended to limit the present invention. Indeed, those skilled in the art will recognize that other alternative hardware and/or software environments may be used without departing from the scope of the invention.

In any event, and with reference now to FIG. 6, after the image 79 is acquired and/or loaded (Block 80 of FIG. 4A), the image 79 may be presented on a display 108 (FIG. 5), such as a computer monitor or a smart phone screen. According to some embodiments of the present invention, loading the image 79 and analysis thereof may be carried out in an APP. 104. A screen capture 100 of an exemplary APP. 104 is shown in said FIG. 6.

The APP. may include a plurality of functionalities, including executing a command to load an image 79 saved in the memory 94 (FIG. 5) of the computing device 84 (FIG. 5) (Icon 112), executing a command to launch a digital camera software (Icon 114), and an image display region 116.

With reference now to FIGS. 4A and 6, and with the image 79 loaded, a region of interest (illustrated as a square 118) within areas of the image 49 representing each of the prepared samples is selected (Block 120). While the illustrated regions of interest 118 is square, it would be readily appreciated by those of ordinary skill in the art that any two dimensional shape (including regular shapes, such as circle or polygon, or irregular shapes) may be used. An area of the region of interest 118 may be resized (Icon 122) or moved within the image 79, such as by fine control movement arrows 124a, 124b, 124c, 124d.

Turning now to FIG. 4B, with continued reference to FIGS. 4A and 6, a flowchart 126 illustrating a method of analyzing a color of a prepared sample is described. The color analysis is described in greater detail in International Patent Application No. PCT/US2013/024622, entitled METHOD AND SYSTEM FOR ANALYZING A COLORIMETRIC ASSAY, filed Feb. 4, 2013, the disclosure of which is incorporated herein by reference in its entirety. Briefly, and after the region of interest 118 is positioned within a first prepared sample (Block 120), an average red value (“R”), green value (“G”), and blue value (“B”) may be determined (Block 128) for the first prepared sample. One exemplary method of calculating the average RGB values may include an incremental averaging technique, such that:

$${AVE}_{N} = {AVE}_{N - 1} + ( \frac{{Pixels}_{N} - {AVE}_{N - 1}}{N} )$$

wherein N is a number of pixels, AVE_{N} is the average value of N pixels, and AVE_{N-1} is the iteratively previous average. The RGB color space average (“RGB”) may then be converted to a CIExyY color space. In that regard, AVE_{N}=C_{srgb}, and linear RGB values may be determined by:

$${C}_{linear} = {( \frac{{C}_{sRGB} + 0.055}{1.055} )}^{2.4}$$

Conventionally, RGB values of images to quantify colors have several limitations. Therefore, the RGB values may be converted to CIE color space coordinates (Block 130). In that regard, the x-, y-, and z-values of the CIE color space may be determined by:

$$\vert \begin{matrix}X \\ Y \\ Z\end{matrix} \vert = \vert \begin{matrix}0.4124 & 0.3576 & 0.1805 \\ 0.2126 & 0.7152 & 0.0722 \\ 0.0139 & 0.1192 & 0.9505\end{matrix} \vert \vert \begin{matrix}{R}_{linear} \\ {G}_{linear} \\ {B}_{linear}\end{matrix} \vert$$

with x- and y-chromaticity values (illustrated as “CIE Coordinates”) determined according to:

$$x = \frac{X}{X + Y + Z} y = \frac{Y}{X + Y + Z}$$

After the x- and y-chromaticity values are determined (Block 130), the chromaticity values of the test sample are compared with the chromaticity values of the LPC and HPC. In that regard, and if the chromaticity values of the test sample are within a range defined by the chromaticity values of the LPC and HPC (“YES” branch of decision block 132), then the chromaticity values of the test sample are considered to be within the range of positive values for the presence of the desired analyte and a “POSITIVE” result is returned (Block 134). Otherwise (“NO” branch of decision block 132), the chromaticity values of the test sample are not within the range of positive values for the presence of the desired analyte and a “NEGATIVE” result is returned (Block 136). If desired, the result may be presented in the APP. 110 as a value 138 (Block 140 of FIG. 4A).

Evaluation of the chromaticity values may include, for example, a Cartesian plot of the x- and y-chromaticity values for the LPC and HPC, with the x- and y-chromaticity values of the test sample overlaid thereon. Alternatively, a linear plot of the a-chromaticity values (or the y-chromaticity values), alone, for the LPC and HPC, with the x- and y-chromaticity values of the test sample overlaid thereon.

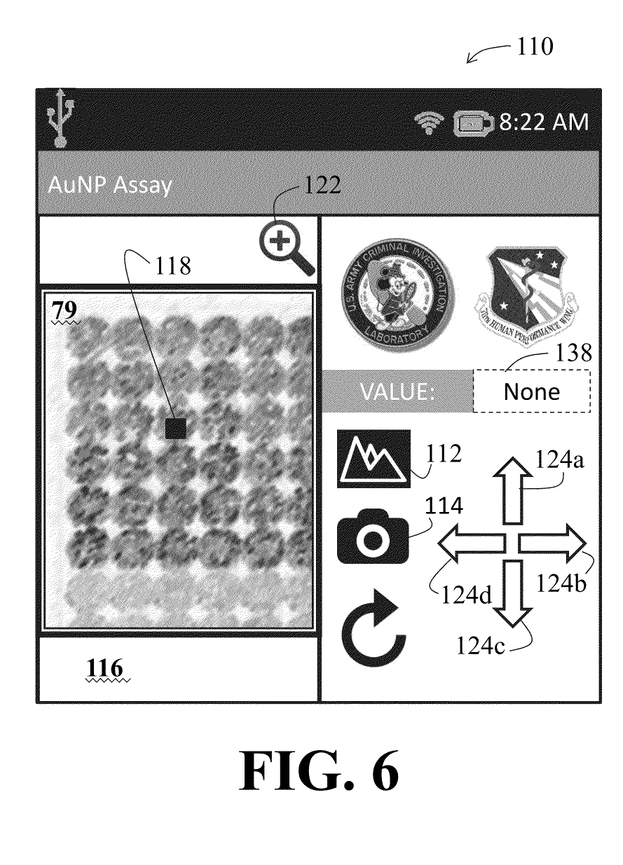

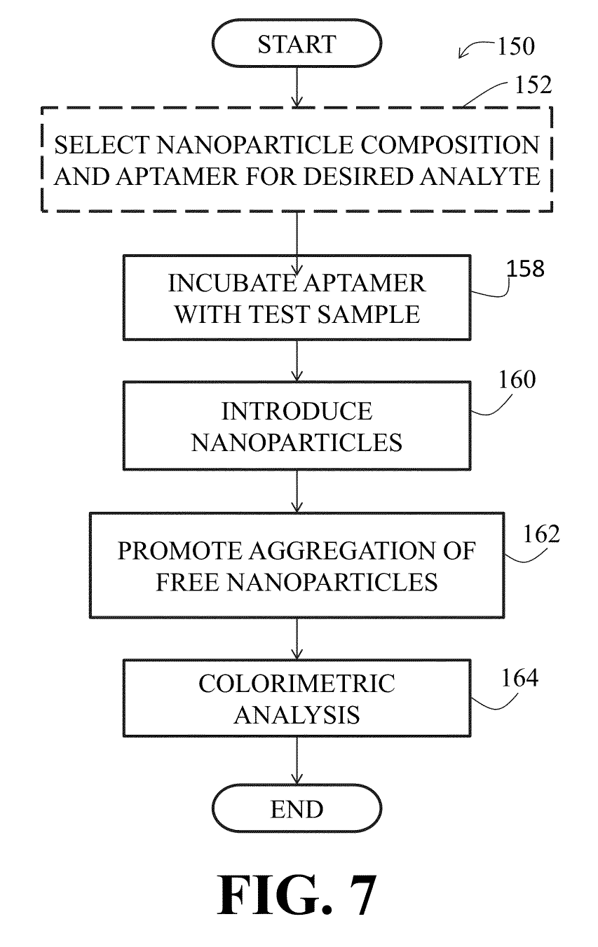

Turning now to FIG. 7, a flowchart 150 illustrating a method of using gold nanoparticle-aptamer conjugates for detection of a desired analyte (or other small molecules) according to another embodiment of the present invention is shown. Again the method starts with optionally selecting a composition for nanoparticles and an aptamer (Block 152), which may be similar to the selection of FIG. 1. For purposes of illustration, the selected aptamer 154 (FIG. 8A) the method of FIG. 7 is described herein as applied to the use of the CBA, MN4 (SEQ ID NO. 1) (molecular weight being 11,128.3 Da). The MN4 aptamer (SEQ ID NO. 1) has been shown, for example, by NMR data, to adopt a folded structure with three stems. Cocaine binds at the junction of the three stems, which is stabilized by noncanonical GA base pairs. Accordingly, MN4 (SEQ ID NO. 1) is structured in a first conformation (or free state) 154 (FIG. 8A) and a second conformation (or bound state) 156 (FIG. 8B) when binding to the analyte (here, cocaine).

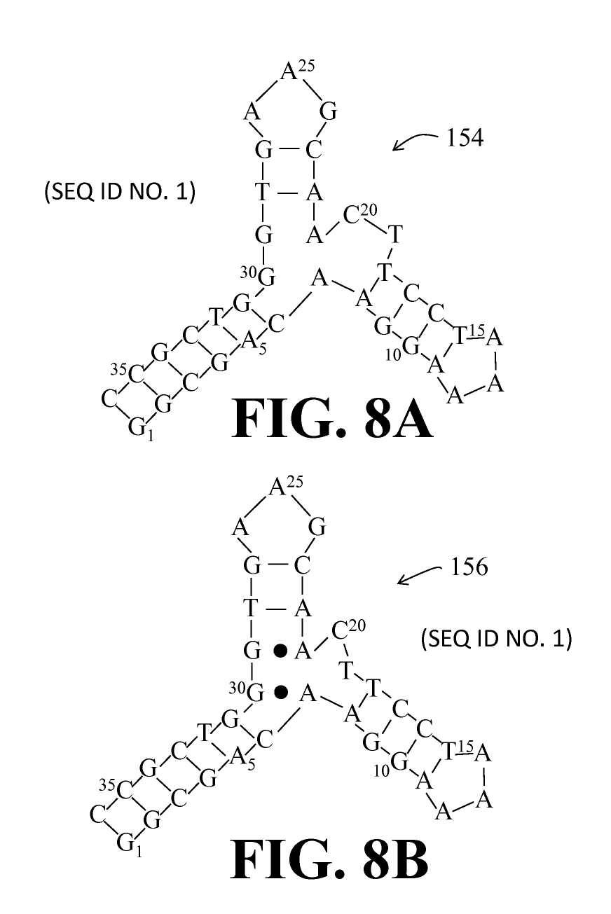

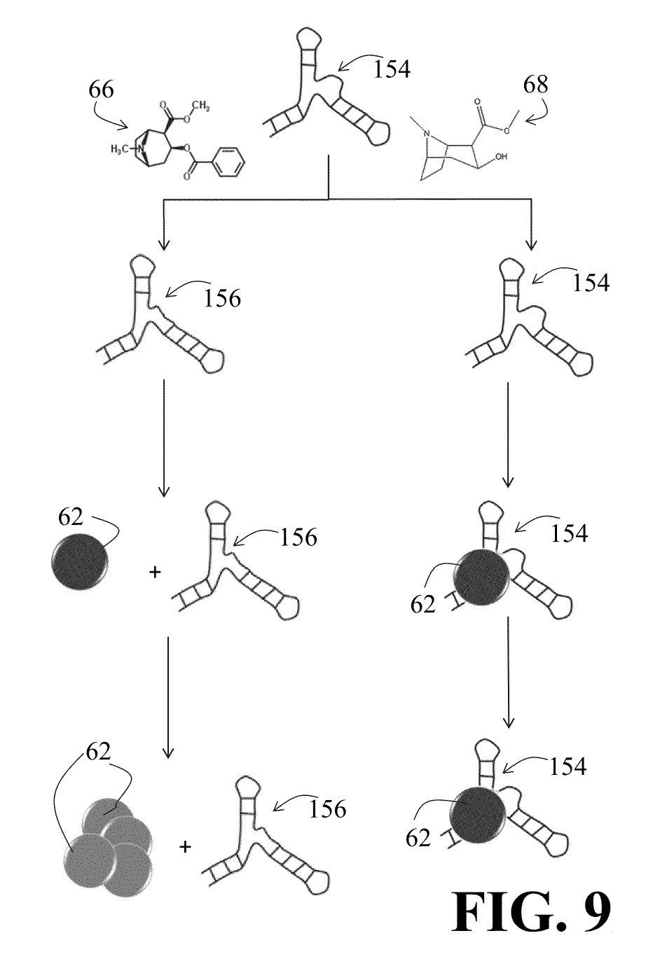

In accordance with block 158 of FIG. 7, and with reference now to FIG. 9, the selected aptamer 154 is incubated with a test sample, which may comprise a biological sample acquired from a patient, an unknown chemical entity, a positive control comprising the analyte of known concentration, or a negative control comprising a known chemical makeup and concentration and without the analyte. For purposes of illustration the unknown chemical entity and the positive control are represented as the cocaine chemical structure 66 in FIG. 9, and the negative control is represented as the EME chemical structure 68 in FIG. 9, wherein EME is a cocaine metabolite. The biological sample and the unknown chemical entity may at least partially comprise the desired analyte 66 such that, if present, would be detected. The biological sample may include, for example, urine, saliva, blood, hair, or sweat. If necessary, the biological sample may be prepared in accordance with a sample preparation protocol or used as collected.

After an incubation time, the selected nanoparticles 62 may be introduced to the test samples (Block 160). The test solution may, optionally, incubate for a time, for example, more than 4 hrs. If the analyte 66 is present, then during the incubation time, the aptamer 156 may undergo a conformational change so as to selectively bind the analyte 66, and as is shown in the test sample branch of FIG. 9. Otherwise, and as shown in the negative control branch of FIG. 9, when no analyte 66 is present, the aptamer 154 does not undergo the conformational change and binds to the nanoparticle 62. In some embodiments, a ratio of bound aptamer to nanoparticle may be greater than 60 to 1.

In block 162 of FIG. 7, aggregation of the free nanoparticles (that is, nanoparticle having no bound aptamer) is promoted. As noted previously, aggregation may occur by masking charges of the nucleic acids comprising the aptamer by introducing a salt to the test solution such that the ionic strength of the test solution is increased. As shown in FIG. 9, free nanoparticles aggregate (as shown in the test sample branch of FIG. 9), which may be perceived as a color change. The color change may be, for example, a visible color; however, any detectable change in wavelength (infrared, ultraviolet, etc.) may also be appropriate. Nanoparticles 62 in solutions comprising nanoparticle bound aptamer (as shown in the negative control branch of FIG. 9), resist aggregation and no color change is perceived.

According to still other embodiments, and as shown in FIG. 9A, a colorimetric response may be promoted without significant conformational change between the nanoparticle bound and unbound states of the aptamer in the presence of the analyte. For example, an aptamer 170 (here, MN4 (SEQ ID NO. 1)) in a first unbound conformation is folded with three stems. In the presence of the analyte 66 (here, cocaine), the MN4 (SEQ ID NO. 1) 170 in the first unbound conformation may undergo a conformational change such that base pairs 176 bind with cocaine 66, as represented by the bound conformation of MN4 (SEQ ID NO. 1) 174. In the presence of the nanoparticles 62, the first unbound conformation further unfolds to a second unbound confirmation of MN4 (SEQ ID NO. 1) 172; however, addition of cocaine 66 still yields the bound conformation of MN4 (SEQ ID NO. 1) 174.

Presence of the color change, or quantification of the amount of color change, may then be determined by a colorimetric analysis (Block 164), which may be carried out as discuss previously.

As described herein, embodiments of the present invention are directed to colorimetric assays for in-the-field use and in which a substantial conformational change between the unbound and bound state is not critical to promote a colorimetric response. Moreover, according to embodiments of the present invention, experimental parameters (as demonstrated here with temperature) can be tuned to allow the aptamer to work in these colorimetric sensors without the need for sequence mutations

### EXAMPLES

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

As to the examples, all the materials were purchased as analytical grade and used without further purification from Sigma-Aldrich (St. Louis, Mo.) unless otherwise indicated. Standard 1 mg/mL methanol solutions of ecgonine methyl ester hydrochloride (“EME”) and cocaine hydrochloride were purchased from Lipomed Inc. (Cambridge, Mass.). DNAse/RNAse Free water and Quant-iT® OliGreen® ssDNA Reagent and were purchased from Invitrogen Corporation (Carlsbad, Calif.). HEPES buffer was purchased from Amresco Inc. (Solon, Ohio).

Test samples were obtained from the United States Army Criminal Investigation Laboratory (USACIL—Forest Park, Ga.). Centrifuge tubes were purchased from Axygen, Inc. (Union City, Calif.).

The aptamers were purchased from Integrated DNA Technologies, Inc. (Coralville, Iowa). DNA batches were purified by standard desalting. The aptamer sequences were 5′-GGC GAC AAG GAA AAT CCT TCA ACG AAG TGG GTC GCC-30 (long cocaine-binding aptamer, MN4 (SEQ ID NO. 1)) and 50-GAC AAG GAA AAT CCT TCA ATG AAG TGG GTC-3′ (short cocaine-binding aptamer, MN6 (SEQ ID NO. 2))

### Example 1

Gold nanoparticles (“AuNPs”) were synthesized by heating and refluxing a 100 mL solution of 1 mM HAuCl₄ at its boiling point with stirring and adding 10 mL of a 38.8 mM sodium citrate solution. The solution continued to boil with mixing for a time (20 min to 25 min), was cooled to room temperature, kept in the dark, and filtered using a 250 mL Corning Filter System with 0.22 μm pore size. The sample was stored at room temperature, wrapped in aluminum foil until used.

The AuNPs were determined to be 15 nm in diameter by dynamic light scattering (“DLS”) using a Zetasizer Nano-instrument (Malvern Instruments, Westborough, Mass.) in backscatter mode (173° detection angle) with the temperature set at 20.0±0.1° C. A final AuNP concentration was determined to be 10 nM using a Cary 300 UV-VIS spectrophotometer (Agilent Technologies, Santa Clara, Calif.) based on the extinction measured at 520 nm, using ε=2.4×10⁸ Lmol⁻¹cm⁻¹.

7.5 mL AuNPs were diluted with 7.5 mL of 20 mM HEPES (HydroxyEthyl PiperazineEthaneSulfonic acid) 2 mM MgCl₂ pH 7.4 buffer in a 50 mL conical vial, and stored in the dark at room temperature, overnight.

Test samples comprising cocaine and control samples comprising ecgonine methyl ester hydrochloride (EME, a structurally related cocaine metabolite) were prepared by adding 1.8 μL of 30 μM CBA (cocaine-binding aptamer: MN4(SEQ ID NO. 1)-AuNPs, MN6(SEQ ID NO. 2)-AuNPs), dissolved in water, to 18.2 μL of the desired concentration of the cocaine or EME dissolved in buffer. After incubation for 30 min, the samples were added to 180 μL of the buffer treated AuNPs and incubated for 30 min. Under these conditions, the aptamers were loaded at a DNA/AuNP ratio of 60, as determined by the method described in Example 3. Finally, NaCl was added to promote AuNP aggregation followed by quantification of the color. Typical NaCl concentrations used in the assay were 25 mM and 40 mM for MN4 (SEQ ID NO. 1) and MN6 (SEQ ID NO. 2), respectively. The exact NaCl concentration varied slightly on a daily basis and was adjusted to obtain consistent background values.

Assay response was monitored by measuring AuNPs extinction in a Spectra Max M5 plate reader (Molecular Devices, Sunnyvale, Calif.) at wavelengths of 650 nm (blue color indicating aggregated AuNPs) and 530 nm (red color indicating dispersed AuNPs) 150 sec after NaCl addition. The resultant adsorption data was plotted as the ratio of aggregated-to-dispersed AuNPs (E650/E530, blue/red), and a calibration curve was obtained. Standard deviation (“SD”) was used to represent the error in the measurements of four replicates, and detection limit (“DL”) was calculated as three times SD above the blank for all calibration curves. DL was used as a measure of the sensitivity of the assay. The data was normalized to a blank for ease of comparison and to account for batch variations.

Images of the test and control samples were acquired 150 sec after the addition of NaCl with a Canon SLR camera (Canon, Inc., Ohta-ku, Tokyo) and a smartphone (Nexus 4, Google, Inc., Mountain View, Calif.) having an 8 MP back-side camera with LED flash.

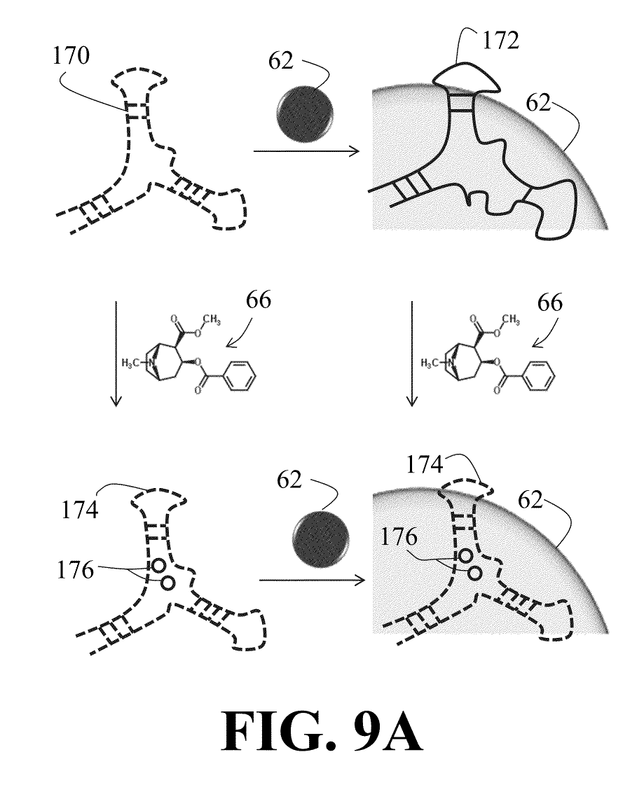

As shown in FIG. 10, MN6(SEQ ID NO. 2)-AuNP samples demonstrated a colorimetric response to cocaine and no response to EME. Despite the predicted lack of conformational change, MN4(SEQ ID NO. 1)-AuNP samples also demonstrated a colorimetric change with the presence of cocaine, but had no response to EME.

To the naked eye, the samples exhibited typical colors observed in the cocaine colorimetric assay.

The data in Table 1, below, illustrates that MN6 (SEQ ID NO. 2) samples provided a better DL than MN4 (SEQ ID NO. 1) samples, although both MN6 (SEQ ID NO. 2) and MN4 (SEQ ID NO. 1) samples showed saturation at similar cocaine concentrations.

Quantification of DNA associated with the AuNP in each sample demonstrated that MN4 (SEQ ID NO. 1) samples had a coating density of only 41 DNA/AuNP. MN6 (SEQ ID NO. 2) samples exhibited higher coating densities after a 30 min adsorption incubation period.

TABLE 1

| | Detection | Assay | |
|---|---|---|---|
| Aptamer | Limit | Saturation | DNA/AuNP |
| MN6 | 1.5 μM | ~75 μM | 56.9 ± 0.9 |
| (SEQ ID NO. 2) | | | |
| MN4 | 5.7 μM | ~75 μM | 40.8 ± 1.7 |
| (SEQ ID NO. 1) | | | |

### Example 2

CBAs were mixed and incubated in buffer with AuNPs (DNA/AuNP ratios of 60 and 300) such that the CBAs adsorbed onto the AuNPs. In that regard, CBA-60-AuNP samples were prepared by mixing 7.5 mL of AuNPs (synthesized according to the method described in Example 1) with 45 μL of 100 μM CBA, dissolved in water, in a 50 mL conical tube. The samples were incubated for 4 hr to 5 hr, and then 7.5 mL of 20 mM HEPES 2 mM MgCl₂ pH 7.4 buffer was added. CBA-300-AuNPs were prepared by mixing 22.5 μL of 1000 μM CBA, dissolved in water, with the AuNPs in a similar manner.

Test samples and controls were evaluated by adding 20 μL aliquots of cocaine (the analyte of interest), one or more controls substances, or both to be tested, prepared in buffer or methanol, to 180 μL of the CBA-60-AuNP and CBA-300-AUNP samples. After incubation for about 1 min, NaCl was added to induce the color change. Typical NaCl concentrations used in the assay were 75 mM and 130 mM for MN4 (SEQ ID NO. 1) and MN6 (SEQ ID NO. 2), respectively. The exact NaCl concentration varied slightly on a daily basis and was adjusted to obtain consistent background values. Data treatment and collection were the same as the methods described in Example 1 and are summarized in FIG. 11 and Table 2 (below).

TABLE 2

| | Detection | Assay | |
|---|---|---|---|
| Aptamer | Limit | Saturation | DNA/AuNP |
| MN6 | No response | — | 58.3 ± 0.1 |
| (SEQ ID NO. 2) | | | |
| MN4 | 15 μM | ~75 μM | 59.0 ± 0.1 |
| (SEQ ID NO. 1) | | | |

### Example 3

The quantity of CBA associated with each AuNP was determined using the Quant-iT® OliGreen® ssDNA Reagent and Kit (Life Technologies, Carisbad, Calif.). Fluorescence intensity of different dye-aptamer mixtures were measured and fit to a calibration curve following the manufacturer's specifications.

For samples prepared in accordance with the methods of Example 1, 500 μL of the CBA-AuNPs were centrifuged using an Amicon® filter (EMD Millipore Corp., Billerica, Mass.) having a molecular weight cut off of 50 kDa. Free DNA passed through the filter, and the filtrate was used to determine the amount of free DNA (that is, DNA that is not associated with, or adsorbed onto, the AuNPs).

For samples prepared in accordance with the methods of Example 2, the samples were prepared in a similar manner; however, the volumes were adjusted to account for dilutions introduced when preparing the assay.

Measurements were performed in duplicate.

### Example 4

Aggregation of NPs was investigated using Transmission Electron Microscope (“TEM”) images, which were acquired with a Hitachi H-7600 TEM (Hitachi Ltd., Tokyo, Japan) on 200 mesh copper grids with a Formvar Carbon film (Electron Microscopy Sciences, Hatfield, Pa.).

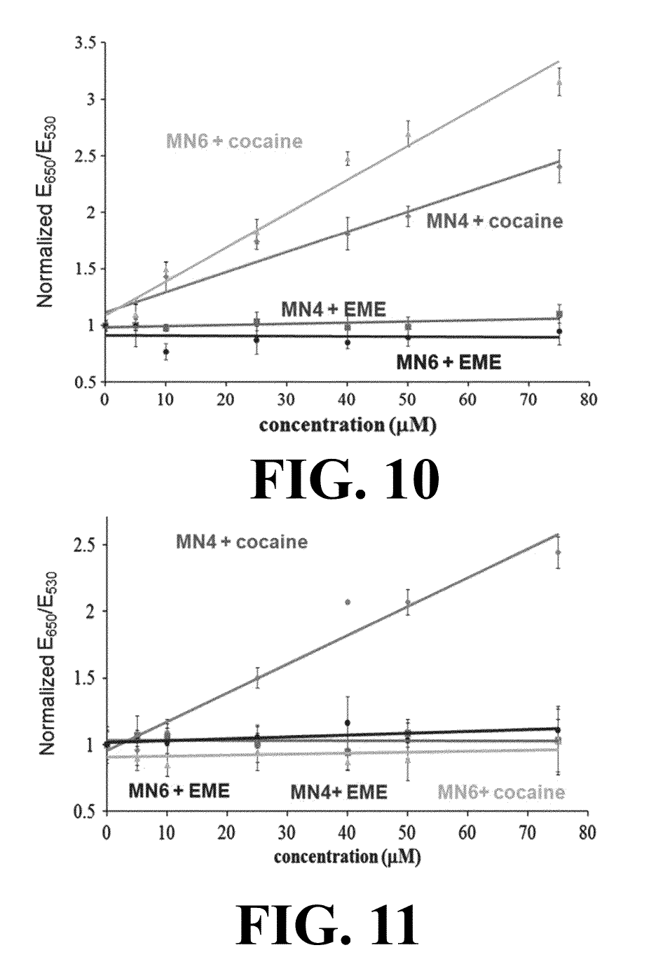

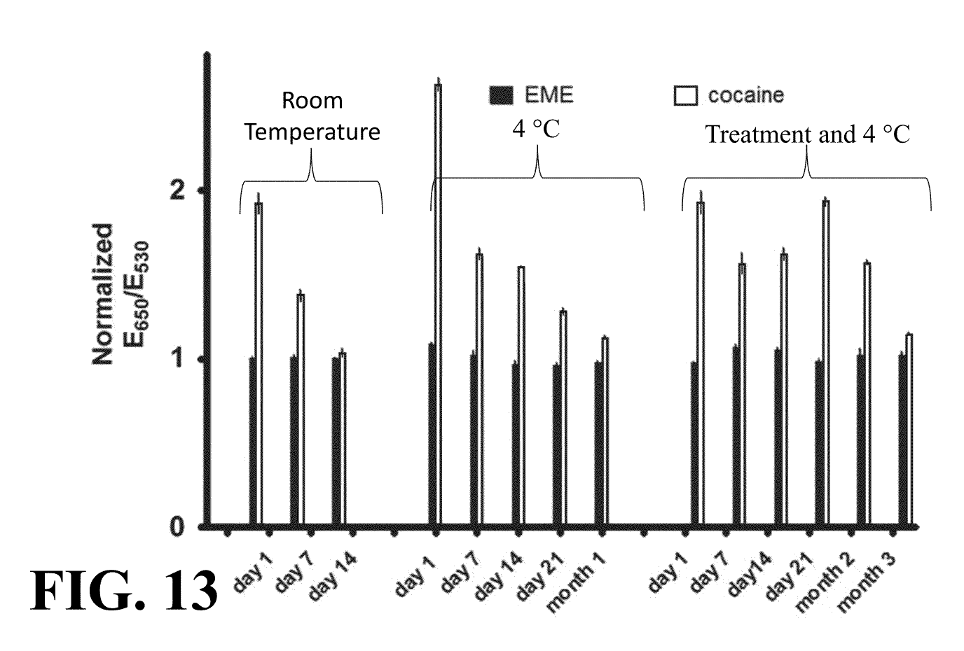

FIGS. 12A and 12B are exemplary TEM images of MN4(SEQ ID NO. 1)-AuNPs challenged with cocaine and EME after NaCl addition, respectively. FIG. 12C is a TEM image of MN4(SEQ ID NO. 1)-AuNPs with no salt added, and FIG. 14D is a TEM image of MN4(SEQ ID NO. 1)-AuNPs exposed to JWH-018 (a synthetic cannabinoid) after NaCl addition.

The images of FIGS. 12A and 12B show typical aggregation in the presence of the analyte (cocaine) but not when exposed to EME (the metabolite). MN6 (SEQ ID NO. 2) did not respond to cocaine; MN4 (SEQ ID NO. 1) responded to cocaine with no response to EME.

### Example 5

Shelf-life of the assays, with and without preservatives, was investigated. To do this, MN4(SEQ ID NO. 1)-AuNPs were treated, overnight, with diethylpyrocarbonate (“DEPC”), autoclaved, and stored at 4° C. until used. The assay response was monitored at different time points for a period of up to 4 mos by dissolving 1 mg/mL of EME (for controls) or cocaine (for tests) in 20 μL aliquots of methanol at a final concentration of 300 μM.

Methanol was used as a target solvent in our “field testing” validation experiments for improved solubility of free-base and hydrochloride analytes while reducing the solubility of inorganic salts. Methanol was used for improved solubility of free-base and hydrochloride analytes while reducing solubilities of inorganic salts, and was shown to not significantly change the assay background levels.

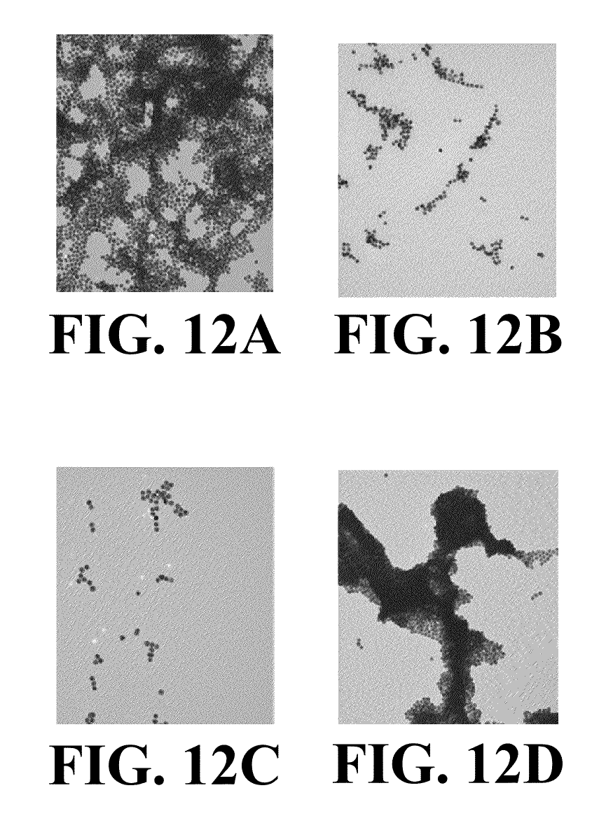

FIG. 13 is a graphical representation of the MN4(SEQ ID NO. 1)-AuNP response to EME and cocaine after treatment to extend shelf-life. A first grouping of data, bracketed as “Room Temperature,” demonstrates the assay response of MN4(SEQ ID NO. 1)-AuNPs to cocaine and EME prepared with AuNPs having no post-synthesis treatment (no DEPC-autoclave) and stored at room temperature, wherein MN4(SEQ ID NO. 1)-AuNPs failed to respond to cocaine after two weeks (as observed by signal decay to below the DL).

A second grouping of data, bracketed as “4° C.,” demonstrates the assay response of MN4(SEQ ID NO. 1)-AuNPs to cocaine and EME prepared with AuNPs having no post-synthesis treatment (no DEPC-autoclave) and stored at 4° C., wherein the shelf-life was shown to be increased to about one month.

A third grouping of data, bracketed as “Treatment and 4° C.,” demonstrates the assay response of MN4(SEQ ID NO. 1)-AuNPs to cocaine and EME prepared with AuNPs having the DEPC-autoclaved treatment and stored at 4° C., wherein the shelf-life was shown to be increased to about two months. It is believed that the extended shelf-life of the third grouping was due to DNase growth prevention by DEPC treatment and the increased AuNPs stability by 4° C. storage.

### Example 6

Specificity of the assay with respect to conventional street cutting agents (including, for example, lactose, sodium bicarbonate, and flour) and other over-the-counter drugs (including, for example, procaine, diphenhydramine (“DPHA”), benzocaine, and lidocaine), collectively referred to as “alternate substances,” was evaluated in a manner similar to the method set forth in Examples 1 and 2 and using a concentration of 1 mg/mL.

Samples prepared in accordance with the method outlines in Example 2 (having MN4(SEQ ID NO. 1)-AuNPs) were tested at the U.S. Army Criminal Investigation Laboratory (Forest Park, Ga.) with alternate substances, as set forth in Table 3 (below), wherein a designation of “+” indicates a positive colorimetric response, a designation of “−” indicates no colorimetric response, and a designation of “+, min” indicates that a slight color change was observed. Each sample was evaluated by naked-eye analysis and with no instrumentation involved.

Briefly, the procaine, DPHA, benzocaine, and lidocaine samples demonstrated a color change prior to NaCl addition even though none have chemical structures similar to cocaine, apart from having amino groups that can interact with the AuNPs surface. FIG. 14 summarizes the data and demonstrates that when AuNPs were exposed to the alternate substances but not to CBAs, the observed change in color was more dramatic (intermediately shaded bars) than when the AuNPs were exposed to MN4 (SEQ ID NO. 1) (darkly shaded bars). The relation suggests that CBAs add stability to AuNPs and may prevent some of interactions with the alternate substances. Moreover samples including MN6 (SEQ ID NO. 2), the CBA having the higher surface affinity, demonstrated an even larger stabilization effect (lightly shaded bars) and a lower response intensity of the false positives.

As shown in FIG. 15, the MN4(SEQ ID NO. 1)-60AuNP samples did not respond to the common household items and sugars tested, which are white powders that could be used as cutting agents. Most substances exhibiting a positive result were controlled substances; however, the stringency of the assay can be tuned to respond preferentially to cocaine by optimizing the DNA coating density.

The MN4(SEQ ID NO. 1)-300AuNP samples did not exhibit response to any of the alternate substances, except JWH-018. In further evaluations, it was determined that color change associated with JWH-018 MN4(SEQ ID NO. 1)-300AuNP samples was due to low solubility, which promoted AuNPs aggregation. The color change due to JWH-018 was distinctly deep blue and easily identifiable as a false positive by a trained eye.

TABLE 3

| | MN4 (SEQ ID | MN4 (SEQ ID |
|---|---|---|
| Alternate Substance | NO. 1)-60 AuNP | NO. 1)-300 AuNP |
| Buffer | − | − |
| Cocaine•HCl | + | + |
| Benzoylecgonine | − | − |
| Ecgonine methyl ester | − | − |
| Heroin•HCl | + | − |
| d-Amphetamine sulfate | + | − |
| MDMA•HCl | + | − |
| Methylone | + | − |
| Methadrone | +, min | − |
| MDPV | + | − |
| Khat | − | − |
| MDMC | + | − |
| Pseudoephedrine•HCl | − | − |
| LSD | + | − |
| JWH-018 | + | + |
| Methamphetamine•HCl | +, min | − |
| Oxycodone•HCl | +, min | − |
| Ketamine•HCl | +, min | − |
| Phentermine•HCl | − | − |
| Benzocaine | − | − |
| Lidocaine | − | − |
| Procaine | + | − |
| HU-210 | − | − |
| Citric acid | − | − |
| Inositol | − | − |
| Lactose | − | − |
| Mannitol | − | − |
| Dextrose | − | − |
| Salicyclic acid | − | − |
| Orange-flavored gelatin | − | − |
| Strawberry-flavored gelatin | − | − |
| YooHoo ® drink powder | − | − |
| Aspirin (acetylsalicyclic acid) | − | − |
| Tide ® detergent powder | − | − |
| AJAX ® detergent powder | − | − |

### Example 7

The algorithm and method described with respect to FIG. 7 were evaluated in an exemplary android-based (OS version 2.3 and higher) color analysis application (“app”) and as compared with conventional plate reader techniques known to those of ordinary skill in the art. In that regard, five replicates of a calibration curve with cocaine standard solutions (0.0 mg/mL, 0.2 mg/mL, 0.4 mg/mL, 0.6 mg/mL, 0.8 mg/mL, and 1.0 mg/mL) were prepared in accordance with the method described in Example 2.

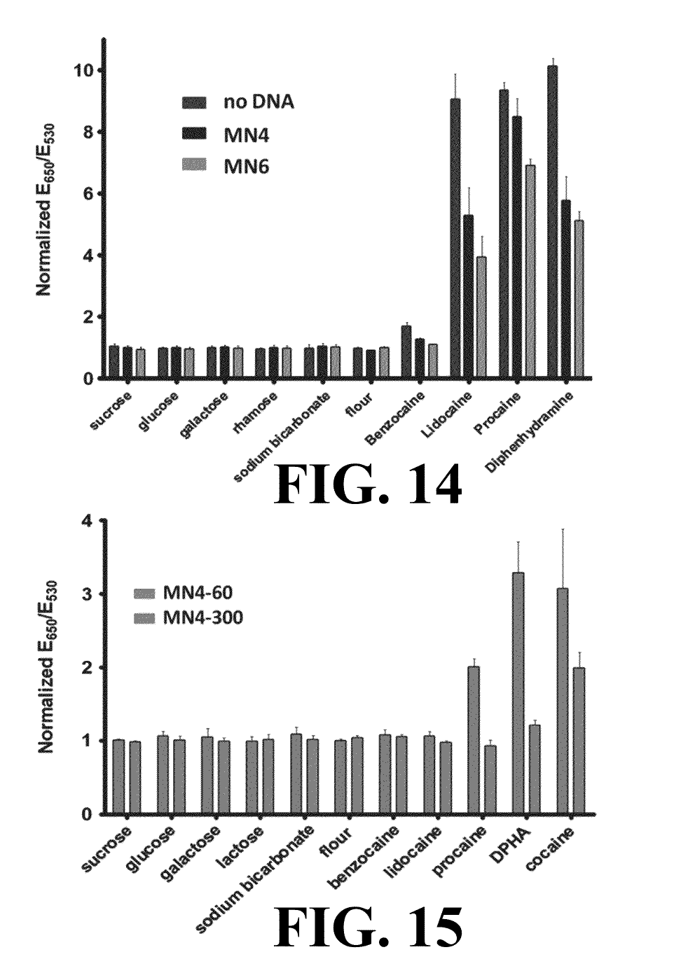

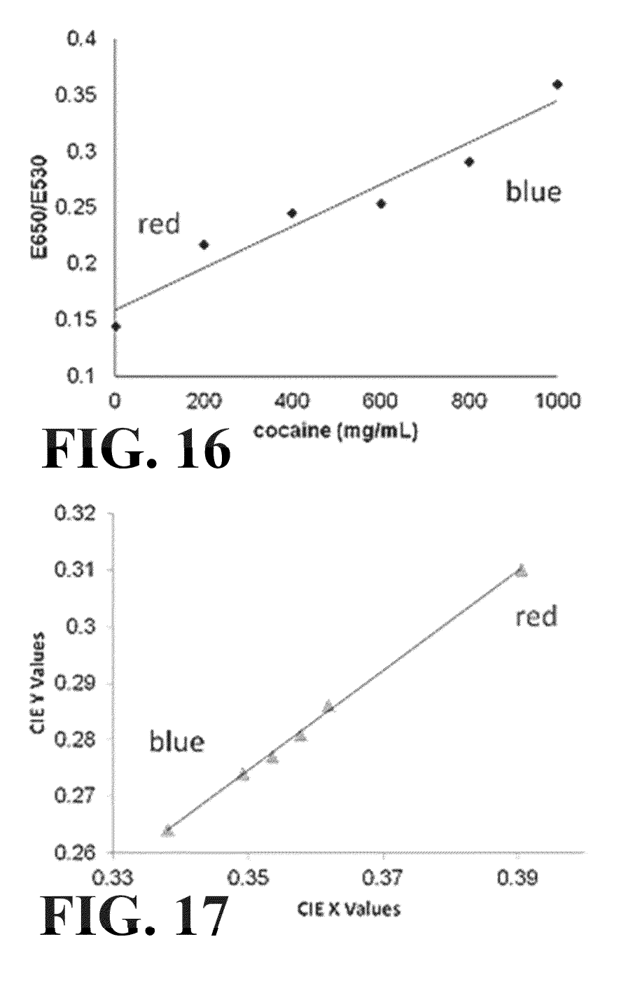

FIGS. 16 and 17 graphically represent calibration curve fits obtained via plate reader and the app, respectively, and demonstrate the validity of the app (y=0.0002x+0.1588 and y=0.8792x+0.0331, respectively). Moreover, when analyzing the same data, the app, which is based on RGB and color map conversion, provided a better fit than the plate reader data (R² of 0.9984 and 0.9385, respectively).

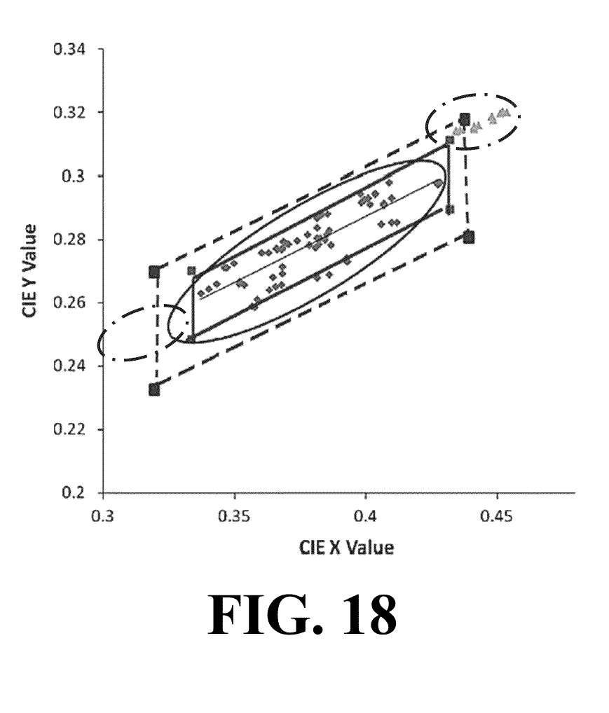

Analysis of data obtained from pictures acquired by the app to set the positive color domain suggested that using both the x- and y-chromaticity values to quantify and compare sample colors was not optimal. For example, and as shown in FIG. 18, use of both coordinates challenged positive/negative boundaries, wherein non-positive values (enclosed with two dash-dot-lined circles) and the positive range (enclosed by a solid-lined oval) often overlapped. The area enclosed with a solid-lined box outlines the range of positive responses, obtained with a certain error value (in this case, one third of the standard deviation, “actual positive response range-1”); however, the solid-lined box does not enclose the whole area given by positive tests (solid-lined oval).

When the positive response area was increased to include all the positive values obtained in the experiments (for example, by utilizing one half of the standard deviation, “actual positive response range-2,” as represented by the dash-lined box), some of the non-positive values were identified as positive (refer to overlapping of dash-lined box and dash-dot-lined circle).

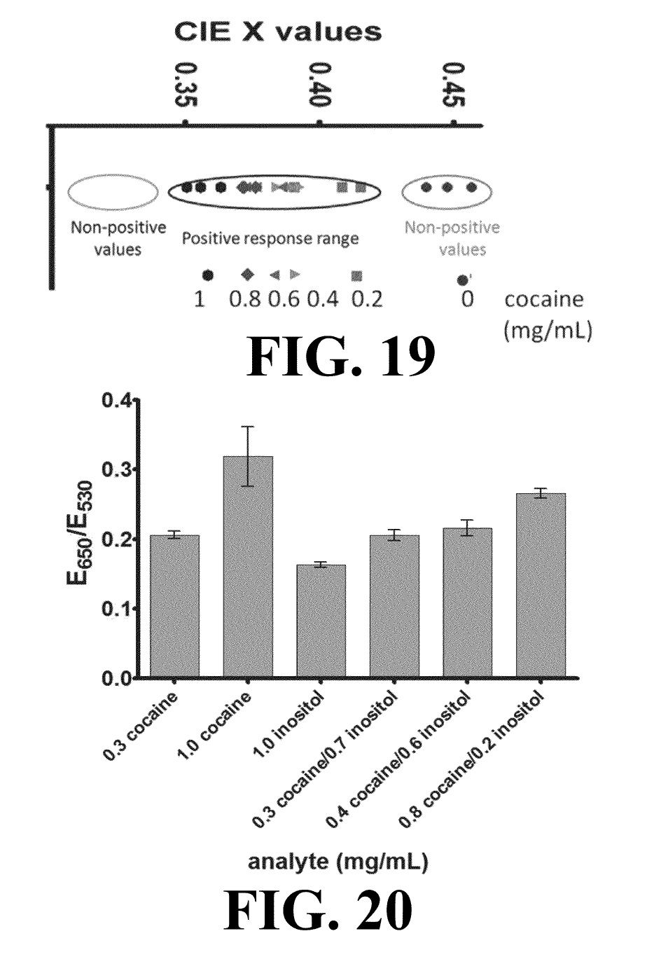

Alternatively, as shown in FIG. 19, a simpler approach for analysis was to one coordinate of the CIE color values for each sample. The CIE y-chromaticity value was observed to result in large variations, making analysis difficult. The CIE x-chromaticity data yielded more reproducible values, reduced the complexity of the data analysis, and maintained the overall trend of the color coordinates. This simplified color analysis offered the option of defining the boundaries of the positive and negative color value intervals in one dimension with minimal overlap.

### Example 8

Validation of the app decision making protocol of Example 7 was performed by testing five replicates of a calibration curve with cocaine standard solutions. The results for each calibration curve were obtained by (1) capturing an image of each of the five replicates and analyzing the color via the app and (2) using conventional plate reader techniques known to those of ordinary skill in the art. Table 4 (below) shows the linear fit of the data obtained via the plate reader and the qualitative analysis via the app.

As to the app, for each replicate, the LPC was set with a blank sample and the HPC was set with a 1.0 mg/mL cocaine standard. Generally, the varying cocaine concentrations were identified as positive by the app.

To show the versatility of the app, the LPC and HPC of each replicate were changed to obtain a narrower positive domain. For example, the LPC and HPC were set using the 0.2 mg/mL cocaine standard and the 0.8 mg/mL cocaine standard, respectively. The app thereafter recognized, as positive, only those standards having cocaine concentrations between more narrowly defined LPC and HPC. Therefore, the blank and the 1 mg/mL cocaine samples were designated as being negative for the presence of cocaine.

Performance of the app was also tested with field-relevant samples, including filler agents and mixtures of cocaine and inositol. The LPC was set to 0.3 mg/mL based on the experimentally determined DL of 0.25 mg/mL. Table 5 (below) shows that the app was successfully in recognized inositol, flour, and lidocaine as non-cocaine substances (that is, returning a “negative” result) at a concentration of 1 mg/mL, the highest possible concentration under the assay settings. On the other hand, mixtures of cocaine with inositol were identified by the app as cocaine-containing samples (that is, returning a “positive” result).

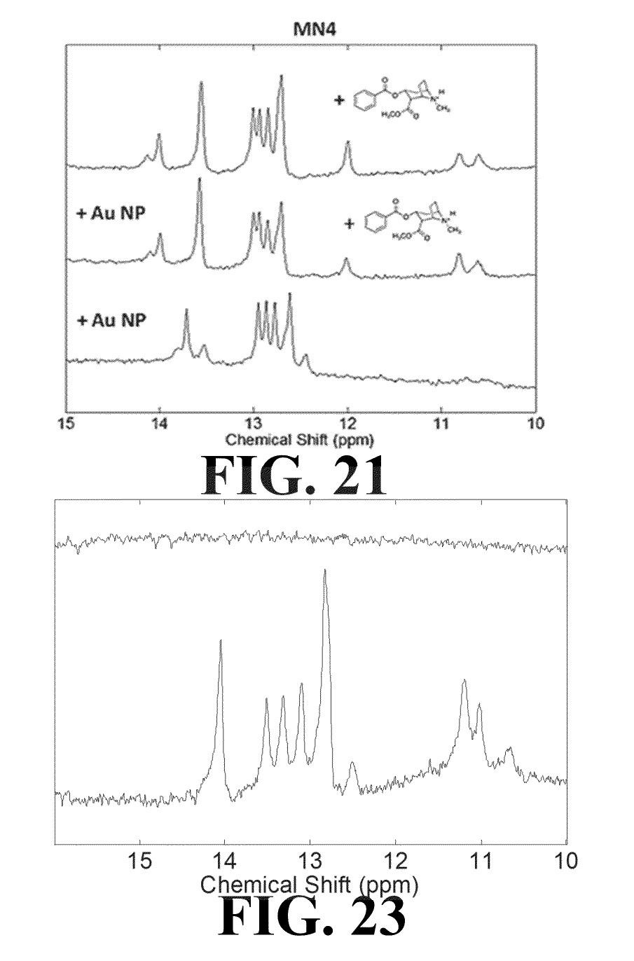

The data presented in FIG. 20 demonstrate that, for samples comprising 30% cocaine with 70% inositol, the plate reader returned a result that was very close to the 0.3 mg/mL cocaine standard (LPC).

TABLE 4

| Concentration | | | | | | |
|---|---|---|---|---|---|---|
| (mg/mL) | 0.0 | 0.2 | 0.4 | 0.6 | 0.8 | 1.0 |
| Plate reader | LPC, + | + | + | + | + | HPC, + |
| calibration 1: | − | LPC, + | + | + | HPC, + | − |
| a = 0.0003, | − | − | LPC, + | HPC, + | − | − |
| b = 0.1558, | | | | | | |
| r² = 0.9525 | | | | | | |
| Plate reader | LPC, + | + | + | + | + | HPC, + |
| calibration 2: | − | LPC, + | + | + | HPC, + | − |
| a = 0.0001, | − | − | LPC, + | HPC, + | − | − |
| b = 0.1314, | | | | | | |
| r² = 0.9505 | | | | | | |
| Plate reader | LPC, + | + | + | + | + | HPC, + |
| calibration 3: | − | LPC, + | + | + | HPC, + | − |
| a = 0.0001, | − | − | LPC, + | HPC, + | − | − |
| b = 0.1787, | | | | | | |
| r² = 0.9264 | | | | | | |
| Plate reader | LPC, + | + | + | + | + | HPC, + |
| calibration 4: | − | LPC, + | + | + | HPC, + | − |
| a = 0.0002, | − | − | LPC, + | HPC, + | − | − |
| b = 0.1588, | | | | | | |
| r² = 0.9385 | | | | | | |
| Plate reader | LPC, + | + | + | + | + | HPC, + |
| calibration 5: | − | LPC, + | + | + | HPC, + | − |
| a = 0.0001, | − | − | LPC, + | HPC, + | − | − |
| b = 0.1246, | | | | | | |
| r² = 0.9752 | | | | | | |
| Plate reader | LPC, + | + | + | + | + | HPC, + |
| calibration 6: | − | LPC, + | + | + | HPC, + | − |
| a = 0.0002, | − | − | LPC, + | HPC, + | − | − |
| b = 0.1417, | | | | | | |
| r² = 0.9613 | | | | | | |

TABLE 5

| Alternate substance | Colorimetric app result |
|---|---|
| Inositol | Negative |
| Inositol | Negative |
| Flour | Negative |
| Flour | Negative |
| Lidocaine | Negative |
| Lidocaine | Negative |
| 30% cocaine + 70% inositol | Positive |
| 30% cocaine + 70% inositol | Positive |
| 40% cocaine + 60% inositol | Positive |
| 40% cocaine + 60% inositol | Positive |
| 80% cocaine + 20% inositol | Positive |
| 80% cocaine + 20% inositol | Positive |

### Example 9

To study the interactions between a CBA (MN4 (SEQ ID NO. 1) or MN6 (SEQ ID NO. 2)), cocaine, and gold nanoparticles, 400 MHz imino NMR spectra were acquired at 297 K. All NMR samples were prepared to a final volume of 500 μL. In NMR tubes, 200 μM DNA-aptamer, 10 mM HEPES, 1 mM MgCl₂ pH 7.4 buffer, and 10% deuterated water were added. Cocaine and EME standards were received from Lipmed at 1 mg/mL dissolved in methanol. The methanol was evaporated using an Eppendorf Vacufuge-Plus (Hamburg, Germany) set at 45° C. and operated for about 1.5 hrs until the samples were dry. Cocaine and EME were each dissolved in 100 μL of deuterated DMSO at a final concentration of 10 mg/mL. To the NMR tubes, either 200 μM cocaine or 2 mM EME was added. For AuNP containing samples, 20 nM AuNPs and 2.5 mM sodium citrate were added to the NMR tubes. The samples were left overnight before performing the NMR experiments, and for the AuNP experiments, cocaine was added moments before obtaining the NMR spectra.

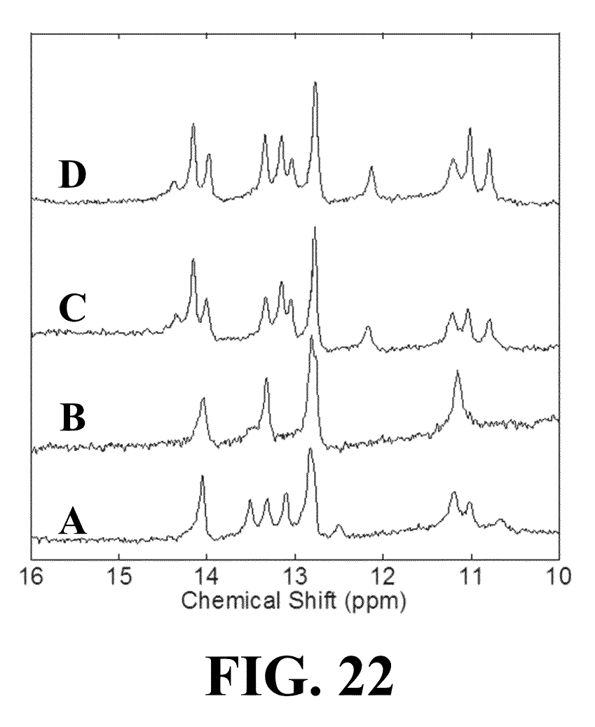

Exemplary spectra with respect to MN4 (SEQ ID NO. 1) are shown in FIG. 21. Significant changes are observed for the MN4 (SEQ ID NO. 1) in the presence of cocaine, which are indicative of a stable complex formation, including well-resolved peaks within the region between 10.5 ppm and 11 ppm (assigned to the GA base pairs) and a large change in a G31 imino peak (12 ppm), as shown in the top line of FIG. 21.

The middle line of FIG. 21 demonstrates significant changes in the spectra as compared to the bottom line of FIG. 21, which correspond to the addition of cocaine to an MN4 (SEQ ID NO. 1) and AuNP mixture. Comparison of the three spectra in FIG. 21 show that the spectrum of the MN4 (SEQ ID NO. 1) and AuNP mixture (bottom line) is nearly identical to the spectrum of the MN4 (SEQ ID NO. 1) and cocaine mixture (top line), which suggests cocaine forms a stable complex with MN4 (SEQ ID NO. 1) while continuing interaction with AuNP.

In another study, NMR analysis of the interactions between MN6 (SEQ ID NO. 2), cocaine, and gold nanoparticles are summarized in FIG. 22. MN6 (SEQ ID NO. 2) differs from MN4 (SEQ ID NO. 1) by the shortening of one stem from six base pairs to three base pairs. The loss of three base pairs destabilizes MN6 (SEQ ID NO. 2), leading to a lower melting temperature and a loss of signal in the imino proton spectra at 297 K, the latter being shown in FIG. 23.

Referring again to FIG. 22, changes in the imino proton signal of MN6 (SEQ ID NO. 2) (line “A” of FIG. 22) are shown with the addition of AuNPs (line “B” of FIG. 22) and with cocaine (line “C”) of FIG. 22). Line “D” of FIG. 22 is a spectrum from the mixture of MN6 (SEQ ID NO. 2), AuNPs, and cocaine, obtained at 273 K.

The addition of AuNP to MN6 (SEQ ID NO. 2) leads to the loss of signals, which are consistent with the fast-exchange limit binding of MN6 (SEQ ID NO. 2) to AuNPs. MN6 (SEQ ID NO. 2) shows large changes in the imino proton signals in the presence of cocaine (line C), consistent with the formation of a stable complex. In the presence of cocaine, the MN6 (SEQ ID NO. 2) spectrum nearly identical spectra are observed in the presence and absence of cocaine.

The quantity of MN4 (SEQ ID NO. 1) associated with each AuNP was analyzed in accordance with the method of Example 3. MN4 (SEQ ID NO. 1) in the presence of gold nanoparticles resulted in an average of 57.3±0.1 aptamers per gold nanoparticle. With the addition of cocaine, an average of 57.4±0.1 aptamers per gold nanoparticle was observed.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

1. A method of detecting a presence of an analyte in a sample, the method comprising:
selecting an aptamer selective to the analyte;
binding the selected aptamer to a nanoparticle, the nanoparticles having a free state perceived as a first color and an aggregate state perceived as a second color;
introducing the sample to the nanoparticle-bound aptamers;
promoting the aggregate state of the nanoparticles; and
analyzing a colorimetric change,
wherein the aggregate state of the nanoparticles is achieved in the presence of the analyte in the sample.

2. The method of claim 1, wherein the aptamer is configured to undergo at least one conformational change when binding to the analyte.

3. The method of claim 1, wherein the nanoparticles comprise gold nanoparticles.

4. The method of claim 1, wherein the aptamer binds to the nanoparticles in the absence of the analyte.

5. The method of claim 1, wherein the analyte is cocaine and the aptamer is MN6 (SEQ ID NO. 2) or MN4 (SEQ ID NO. 1).

6. The method of claim 1, wherein analyzing a colorimetric change further comprises:
loading an image of the sample;
determining a red-value, a blue-value, and a green-value for a portion of the image representing the sample;
converting the red-value, the blue-value, and the green-value to CIE color space coordinates;
determining whether the CIE color space coordinates are within a range defined by a low concentration of the analyte and a high concentration of the analyte; and
based on the determining, returning a positive result for the presence of the analyte when the CIE color space coordinates are within the range, or returning a negative result for the presence of the analyte when the CIE color space coordinates are not within the range.

7. The method of claim 6, wherein the image includes a low concentration control and a high concentration control.

8. The method of claim 7, wherein determining whether CIE color space coordinates are within the range further comprises:
comparing x-chromaticity values of the sample with x-chromaticity values of the low concentration control and x-chromaticity values of the high concentration control.

9. The method of claim 8, further comprising:
comparing y-chromaticity values of the sample with y-chromaticity values of the low concentration control and y-chromaticity values of the high concentration control.

10. A method of detecting a presence of an analyte in a sample, the method comprising:
preparing the sample using an aptamer-nanoparticle conjugate assay;
loading an image of the prepared sample;
determining a red-value, a blue-value, and a green-value for a portion of the image representing the prepared sample;
converting the red-value, the blue-value, and the green-value to CIE color space coordinates;
determining whether the CIE color space coordinates are within a range defined by a low concentration of the analyte and a high concentration of the analyte; and
based on the determining, returning a positive result for the presence of the analyte when the CIE color space coordinates are within the range, or returning a negative result for the presence of the analyte when the CIE color space coordinates are not within the range.

11. The method of claim 10, wherein the aptamer-nanoparticle conjugate assay comprises:
selecting an aptamer selective to the analyte;
mixing the aptamer with nanoparticles such at least one aptamer binds to each nanoparticle, the nanoparticles having a free state perceived as a first color and an aggregate state perceived as a second color;
introducing the sample into the aptamer and nanoparticle mixture;
promoting the aggregate state of the nanoparticles; and
analyzing a colorimetric change,
wherein the aggregate state of the nanoparticles is achieved in the presence of the analyte in the sample.

12. The method of claim 10, wherein the nanoparticles comprise gold nanoparticles.

13. The method of claim 10, wherein the aptamer binds to the nanoparticles in the absence of the analyte.

14. The method of claim 10, wherein the analyte is cocaine and the aptamer is MN6 (SEQ ID NO. 2) or MN4 (SEQ ID NO. 1).

15. The method of claim 10, wherein the aptamer-nanoparticle conjugate assay comprises:
selecting an aptamer selective to the analyte;
mixing the aptamer with the sample;
introducing nanoparticles into the aptamer and analyte mixture, the nanoparticles having a free state perceived as a first color and an aggregate state perceived as a second color;
promoting the aggregate state of the nanoparticles; and
analyzing a colorimetric change,
wherein the aggregate state of the nanoparticles is achieved in the presence of the analyte in the sample.

16. The method of claim 15, wherein the nanoparticles comprise gold nanoparticles.

17. The method of claim 15, wherein the aptamer binds to the nanoparticles in the absence of the analyte.

18. The method of claim 15, wherein the analyte is cocaine and the aptamer is MN6 (SEQ ID NO. 2) or MN4 (SEQ ID NO. 1).

19. An apparatus for detecting a presence of an analyte in a sample, the apparatus comprising:
a camera;
a memory;
a processor;
a display; and
program code resident in the memory and configured to be executed by the processor to acquire a photograph of the sample prepared using an aptamer-nanoparticle conjugate assay and return a result indicating the presence of the analyte, the program code further configured to:
determine a red-value, a blue-value, and a green-value for a portion of the loaded image representing the prepared sample;
convert the red-value, the blue-value, and the green-value to CIE color space coordinates;
determine whether the CIE color space coordinates are within a range defined by a low concentration of the analyte and a high concentration of the analyte;
based on the determining, return a positive result for the presence of the analyte when the CIE color space coordinates are within the range or return a negative result for the presence of the analyte when the CIE color space coordinates are not within the range.

20. A method of detecting a presence of an analyte in a sample, the method comprising:
selecting an aptamer selective to the analyte;
mixing the aptamer with nanoparticles such at least one aptamer binds to each nanoparticle, the nanoparticles having a free state perceived as a first color and an aggregate state perceived as a second color;
introducing the sample into the aptamer and nanoparticle mixture;
promoting the aggregate state of the nanoparticles; and
analyzing a colorimetric change,
wherein the aggregate state of the nanoparticles is achieved in the presence of the analyte in the sample.


SEQUENCE LISTING (2 sequences)

SEQ ID NO: 1 - DNA, 36 bp, Artificial Sequence
  Synthetic aptamer motif sequence
ggcgacaaggaaaatccttcaacgaagtgggtcgcc

SEQ ID NO: 2 - DNA, 30 bp, Artificial Sequence
  Synthetic aptamer motif sequence
gacaaggaaaatccttcaatgaagtgggtc